(19) United States
(12) Patent Application Publication
(10) Pub. No.: US 2005/0137477 A1
(43) Pub. Date: Jun. 23, 2005

(54) Dynamic display of three dimensional ultrasound ("ultrasonar")

(75) Inventor: Ralf Alfons Kockro, Singapore (SG)

Correspondence Address: KRAMER LEVIN NAFTALIS & FRANKEL LLP, INTELLECTUAL PROPERTY DEPARTMENT, 1177 AVENUE OF THE AMERICAS, NEW YORK, NY, 10036, US

(73) Assignee: Volume Interactions Pte. Ltd., Singapore (SG)

(21) Appl. No.: 10/744,869

(22) Filed: Dec. 22, 2003

(51) Int. Cl. A61B 8/00

(52) U.S. Cl. 600/437

(57) ABSTRACT

A method and system for the dynamic display of three dimensional ultrasound images is presented. In exemplary embodiments according to the present invention, the method includes acquisition of a plurality of ultrasound images with a probe whose position is tracked. Using the positional information of the probe, the plurality of images are volumetrically blended using a pre-determined time dependent dissolving process. In exemplary embodiments according to the present invention a color look up table can be used to filter each image prior to its display resulting in real-time segmentation of greyscale values and the three-dimensional visualization of the three-dimensional shape of structures of interest.

### FIELD OF THE INVENTION

The present invention relates to the field of medical imaging, and more particularly to the interactive and real-time display of three-dimensional ultrasound images.

### BACKGROUND OF THE INVENTION

During a conventional medical ultrasound examination an online image of a captured area of interest is displayed on a monitor next to an examiner (generally either a radiologist or an ultrasound technician). The displayed image reflects the plane of the ultrasound image acquisition and is displayed as a flat image in a fixed window on the monitor screen. The refresh rate of such an image is usually greater than 20 frames/second. This conventional method does not offer the ultrasound examiner any sense of three dimensionality, and thus there are no visual cues to provide the examiner with depth perception. The only interactive control an examiner has with the device is which cross-sectional plane to view in a given field of interest. Wherever the ultrasound probe is moved determines which two-dimensional plane the examiner will see. If a user desires to correlate two or more of these two-dimensional planes so as to be able to follow a three dimensional structure across them (such as where the planes of the ultrasound are perpendicular to the longitudinal axis of such a structure), this can only be done mentally.

Alternatively, conventional methods exist for volumetric ultrasound image acquisition. These methods keep track of the spatial position of an ultrasound probe during image acquisition by, for example, tracking the probe with an electromagnetic tracking system, while simultaneously recording a series of images. Thus, using the series of two-dimensional images acquired as well as the knowledge of their proper order (acquired by the tracking device), a volume of the scanned bodily area can be reconstructed. This volume can then be displayed and segmented using standard image processing tools. Since the conventional volumetric reconstruction process can take from 4-30 seconds (depending upon the number of slices captured, the final resolution required and the amount of filtering being done), such a rendered volume cannot be online and thus cannot be dynamically interacted with by a user.

Several manufacturers of ultrasound systems, such as, for example, GE, Siemens, Toshiba and others offer such volumetric 3D ultrasound technology. A similar process is one where no tracking system is used, but a certain speed of scan and movement of the hands—which can be either linear or a sweep—is assumed in reconstructing a volume from the series of 2D scans. In each of these conventional methods the overall process of sweeping, saving the images and converting them to a volume can take from a few seconds, or even a few minutes depending on the hardware, the kind of processing desired on the image, etc.

Typical applications for 3D ultrasound range from viewing the prenatal foetus to hepatic, abdominal and cardiological ultrasound imaging. Additionally, many 3D ultrasound systems, such as those offered, for example, by Kretz (Voluson 730) or Philips (SONOS 7500), restrict the volume that can be captured to the footprint of the probe, thus restricting the volumes that can be viewed to small segments of a body or other anatomical structure. Although a user could acquire numerous probe footprints, it is currently still difficult to save all such volumes due to memory limitations. Therefore, most scanning is “live”, meaning that the data is seen but not stored. Thus, a problem with volumetric probes which do not use a tracking system is that since the probe footprint is spatially limited, when a user moves a probe to another place on a patient's body, it loses the memory of what he saw at the prior location.

Thus, each of the conventional methods described above has certain drawbacks. As described above, the standard ultrasound display technique of online two dimensional images of the ultrasound acquisition plane does not provide any volumetric information. In order to understand the spatial information of a scanned area, a user needs to memorize the flow of the ultrasound images in relation to the position and orientation of the ultrasound probe as well as the direction and speed of the probe's movement. This is usually quite difficult and requires substantial experience. Even with significant experience, many examiners simply cannot mentally synthesize a sequence of images so as to truly see a mental volume reflecting the interior of the actual anatomy being scanned. People who are not highly visual may have difficulty in remembering the previously viewed images so as to mentally superimpose them upon the image in current view. On the other hand, as noted above, it is possible to track the ultrasound probe (such as, for example, using an electromagnetic or optic tracking system) and use that information to subsequently reconstruct the volume accordingly. Nonetheless, such a three dimensional volume is not available online (inasmuch as the generation takes time) and is also static, not being integrated into the dynamic Ultrasound examination process. Since ultrasound is fundamentally a dynamic and user dependent examination, static visualizations—even if volumetric—are undesirable.

What is thus needed in the art is a method for dynamically displaying ultrasound images that is both three dimensional as well as dynamic and interactive, and where an area displayed in dynamic 3D is not restricted to the field of view of an ultrasound probe.

### SUMMARY OF THE INVENTION

A method and system for the dynamic display of three dimensional ultrasound images is presented. In exemplary embodiments according to the present invention, the method includes acquisition of a plurality of ultrasound images with a probe whose position is tracked. Using the positional information of the probe, a plurality of images are volumetrically blended using a pre-determined time dependent dissolving process. In exemplary embodiments according to the present invention a color look up table can be used to filter each image prior to its display, resulting in real-time segmentation of greyscale values and the three-dimensional visualization of the three-dimensional shape of structures of interest.

### BRIEF DESCRIPTION OF THE DRAWINGS

FIG. 1 illustrates a plurality of ultrasound image planes displayed with varying transparency according to an exemplary embodiment of the present invention;

FIG. 2 depicts a process flow chart according to an exemplary embodiment of the present invention;

FIG. 3 illustrates the display of an ultrasound image over a checkerboard background using various opacity values;

FIG. 4 depicts the ultrasound image of FIG. 3 with 100% opacity and an increase in pixel brightness of 50%;

FIG. 5 depicts example “linear opaque” plots of opacity vs. intensity for the four example images of FIG. 3;

FIG. 6 depicts alternative exemplary “customized color look up table” plots of opacity vs. intensity;

FIG. 6A depicts an exemplary opacity vs. intensity plot illustrating a linear color look-up table according to an exemplary embodiment of the present invention.

FIG. 7 depicts the four exemplary displays of FIG. 3, using the opacity vs. intensity curves of FIG. 6;

FIG. 8 is a graphic illustration of a three-dimensional cone scanned with a plurality of sequential ultrasound images according to an exemplary embodiment of the present invention (scan direction is from the left to the right of the figure, i.e., from the opening to the vertex of the depicted exemplary cone);

FIG. 9 depicts the perspective of a viewer of the resulting ultrasound scan images from the exemplary scan illustrated in FIG. 8;

FIG. 10 depicts the first (leftmost) scan of FIG. 8 as the current scan, blended with an exemplary background using a given transparency value;

FIG. 11 depicts the first and second scans of FIG. 8, blended using an exemplary time dependent dissolving algorithm against an exemplary background according to an exemplary embodiment of the present invention;

FIG. 12 depicts the first, second and third scans of FIG. 8, blended using an exemplary time dependent dissolving algorithm against an exemplary background according to an exemplary embodiment of the present invention;

FIG. 13 depicts the first through fourth scans of FIG. 8, blended using an exemplary time dependent dissolving algorithm against an exemplary background using a given transparency value according to an exemplary embodiment of the present invention;

FIG. 14 depicts the first through fifth scans of FIG. 8, blended using an exemplary time dependent dissolving algorithm against an exemplary background using a given transparency value according to an exemplary embodiment of the present invention;

FIG. 15 depicts the first through sixth scans of FIG. 8, blended using an exemplary time dependent dissolving algorithm against an exemplary background using a given transparency value according to an exemplary embodiment of the present invention;

FIG. 16 depicts the first through seventh scans of FIG. 8, blended using an exemplary time dependent dissolving algorithm against an exemplary background using a given transparency value according to an exemplary embodiment of the present invention;

FIG. 17 depicts all eight scans of FIG. 8, blended using an exemplary time dependent dissolving algorithm against an exemplary background using a given transparency value according to an exemplary embodiment of the present invention;

FIGS. 18-25 depict the eight scans of FIG. 8 successively added together, according to an exemplary embodiment of the present invention;

FIG. 26 depicts a top perspective view of a set of example phantom objects used in generating the exemplary images depicted in FIGS. 29 through 34;

FIG. 27 depicts a side perspective view of the exemplary set of phantom objects of FIG. 26;

FIG. 28 depicts exemplary combinations of ultrasound images of the phantom objects depicted in FIG. 27 using various numbers of slices according to an exemplary embodiment of the present invention;

FIGS. 29-33 respectively depict the exemplary combinations of ultrasound images of FIG. 28 wherein the color look-up table and fade rate parameters are varied according to an exemplary embodiment of the present invention;

FIG. 34 depicts an exemplary set of blended ultrasound images with the current image plane in front using a linear color look-up table;

FIG. 35 depicts an exemplary set of blended ultrasound images with the current image plane in back using the exemplary linear color look-up table of FIG. 34;

FIG. 36 depicts an exemplary set of blended ultrasound images with the current image plane in front using an exemplary customized color look-up table;

FIG. 37 depicts an exemplary set of blended ultrasound images with the current image plane in front using the exemplary customized color look-up table of FIG. 36;

FIG. 38 depicts an exemplary two-part system according to an exemplary embodiment of the present invention;

FIG. 39 depicts an exemplary integrated system according to an exemplary embodiment of the present invention; and

FIG. 40 depicts an exemplary external box system according to an exemplary embodiment of the present invention.

### DETAILED DESCRIPTION OF THE INVENTION

An ultrasound examination is a dynamic and user dependent procedure, where a diagnosis is generally obtained during the examination itself and not by a retrospective image analysis. Thus, to be useful, a volumetric display of ultrasound data must be dynamic and in substantially real time.

In exemplary embodiments according to the present invention online volume displays of ultrasound images can be provided to a user using a standard single-plane ultrasound scanner. In exemplary embodiments according to the present invention, ultrasound image data coming out of a scanner can either be redirected to a separate computer with system hardware or software, or hardware and/or software implementing an exemplary embodiment of the invention can be loaded and/or installed into a standard ultrasound machine and process the data prior to display. In preferred exemplary embodiments of the present invention the same ultrasound scanner can house the image producing hardware and a 3D probe tracker. Alternatively, a computer can be added to an ultrasound scanner, and this extra computer can receive the ultrasound images and house the tracker, and can then combine image with tracker information to produce a new display. Because these displays are online (i.e., the displayed data is in substantially real time relative to its acquisition), they can be available to a user, for example, while he or she carries out a dynamic ultrasound examination. Thus, a user can be presented with real-time depth perception that can be constantly updated as the user dynamically moves an ultrasound probe in various directions through a field of interest. This functionality is markedly different from conventional approaches to volumetric ultrasound display in that the presented volume is not restricted to the footprint of an ultrasound probe.

In exemplary embodiments according to the present invention, a volumetric ultrasound display can be presented to a user by means of a stereoscopic display that further enhances his or her depth perception.

In exemplary embodiments according to the present invention, a displayed volume can be constantly updated when dynamically moving the probe in various directions through a field of interest. In such exemplary embodiments the imaging data of the ultrasound probe is displayed in a way which is similar to that of radar or sonar display: the most recent and online image is displayed as opaque and bright, whereas older images turn transparent, or “fade.” The older a given image gets, i.e., the more time that has passed since the image was acquired, the more it fades away. In exemplary embodiments according to the present invention, the three-dimensional position of the ultrasound probe is continually tracked using a tracking system, according to standard techniques as are known in the art. Thus, a displayed three-dimensional volume can be constantly refreshed relative to the then current position of the probe. Moreover, a user can sweep back and forth across a particular surface region so as to view the three-dimensional structures below from different directions, dynamically choosing how the volume of any particular structure of interest is visualized. Using the tracked position of the probe as each image is acquired images acquired at arbitrary positions of the probe can be coherently synthesized.

In exemplary embodiments according to the present invention, the fading speed of noncurrent images can be dynamically adjusted by a user so as to adapt to the dynamics of a given ultrasound examination. Thus, for example, fast back and forth movements of the ultrasound probe over a small area can utilize faster fading rates, whereas slower probe movements can, for example, utilize a slower fading rate.

In exemplary embodiments according to the present invention color coding can also be used to provide a useful visual cue. Thus, the most recent image can, for example, be displayed in its original greyscale and the increasingly aging image planes could be displayed in color, in addition to becoming more transparent with time. In such exemplary embodiments a color look up table can be used to map the noncurrent images' greyscale values to a color of choice. Color choice can be determined by a user, and can include, for example, all one color, or different colors associated with different acquisition times, among various other possibilities. Additionally, in exemplary embodiments of the present invention an indication of the position/orientation/extent of the noncurrent images can be implemented without showing the images themselves, such as, for example, displaying only their outline box, so that a user knows where the images were taken without the images themselves obscuring the display.

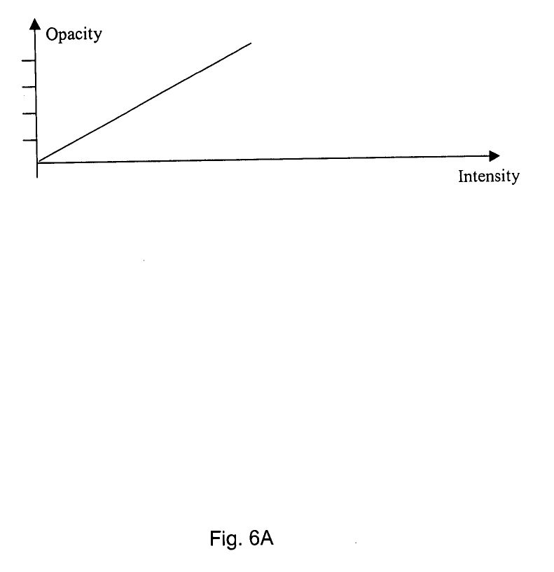

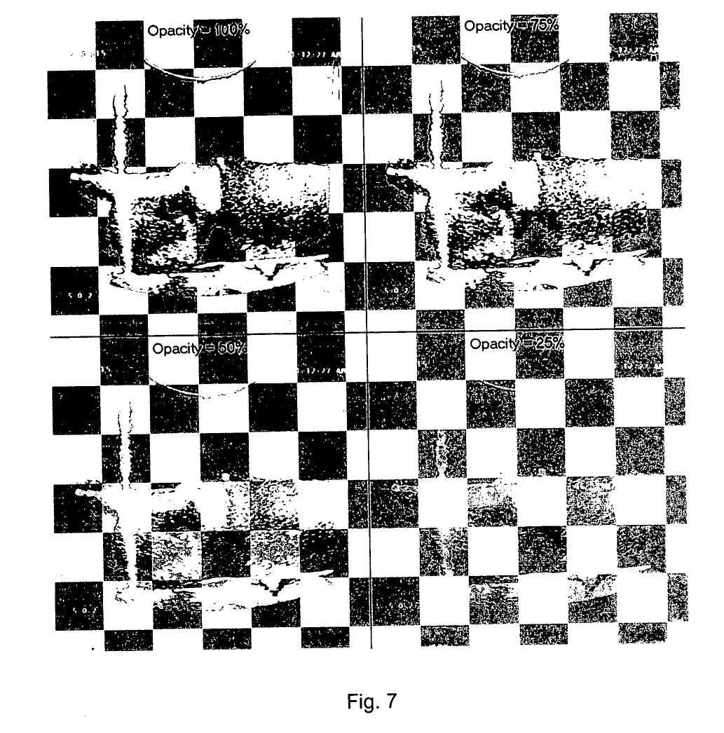

Additionally, in exemplary embodiments according to the present invention a color lookup table can be used to “filter” images prior to display, resulting in real-time segmentation of certain grey-scale values and thus the three-dimensional visualization of the three-dimensional shape of structures of interest. Unwanted parts of an image can thus be filtered out to enhance the perception of the resulting volume. For example, FIG. 6 depicts a number of customized color look up tables (“CLUT”) 610, 620, 630 and 640, corresponding respectively to different opacity values. Using these tables, a black background can be filtered out of an image to reveal the edge of an object. An example of this is depicted in FIG. 7 (assuming a system where an intensity of 0 is black and that of 255 is white, so setting all pixel values below a threshold as transparent precludes the display of blacker pixels). This can be accomplished, for example, by mapping the transparency of certain pixels to be either opaque or transparent (or to any value in between). With reference to FIG. 6, for example, the intensity value 601 is the intensity threshold below which all pixels are displayed as completely transparent for each of CLUTs 610-640.

Additionally, a CLUT can be dynamically modified by a user. A CLUT maps the transparency and color of any value in the image to another value to be displayed on the screen. For example, an original image can provide an index (i.e., the original pixel value, say 8 bits) that can be transformed into a (Red, Green, Blue, or “R,G,B”) 24-bit color value that can be loaded into a graphics card, resulting in a particular color being displayed for that pixel on a monitor. Moreover, a transparency parameter T can also be added, as, for example, another 8 bit value, giving a range of 256 degrees of transparency, thus associating an (R,G,B,T) value with each original pixel in a given image. For example, a tumor which appears as whitish in a given ultrasound image can be isolated from surrounding darker grey pixels so that its three-dimensional shape can be more easily appreciated by a viewer. This can be implemented, for example, by identifying the correct grey scale range of the tumour and setting all neighboring darker values to full transparency. This is described in greater detail below in connection with varying opacity with pixel intensity as illustrated by FIGS. 6, 6A and 7.

In exemplary embodiments according to the present invention, if an ultrasound beam is directed through a given area during an examination which is still represented on a system display by noncurrent (fading) ultrasound images, the online or current image can, for example, overwrite the “older” volume. Thus, as noted, the displayed 3D volume can be constantly refreshed relative to the currently acquired ultrasound image.

It is noted that the functionalities of exemplary embodiments according to the present invention are facilitated by the generation of real-time volumes during sweeps of an ultrasound probe by volumetrically adding up the acquired ultrasound images and by allowing a time dependent transparency change. The details of this process are next described.

Creation of a Volume Effect Using Transparency Blending

The transparency of an image refers to the effect of blending that image with image data originating behind it. By displaying several transparent images superimposed on each other a volumetric effect can be created. The display technique uses back-to-front blending of images. Within each image, areas that are not wanted can be turned transparent (segmented out) to enable a user to visualize regions of interest (such as, for example, a vessel or an organ). Such transparency can be full or semitransparent.

In exemplary embodiments according to the present invention, displaying transparency is not implemented by lowering the brightness of a given pixel in an image (i.e., a pixel in a non-background image), but by lowering the opacity of that pixel. The opacity of a pixel (known in the art as its alpha value) represents its blending strength with its background.

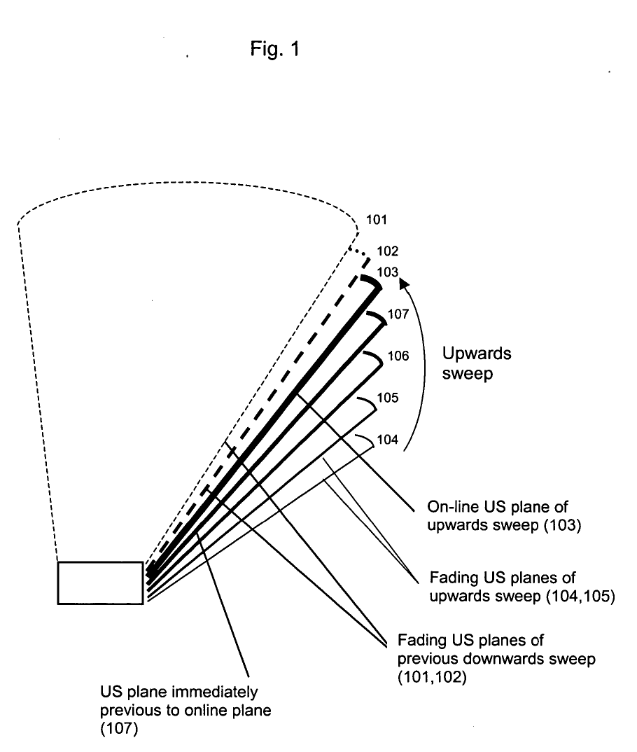

Thus, with reference to FIG. 1, a number of ultrasound image planes are shown. The current or online image plane is 103, and image planes 104 through 107 were acquired prior to it, in that sequence. Ultrasound plane 107 is the immediately prior plane to current plane 103. Thus, in this example, the ultrasound probe has been swept upward from location 104 to location 103. Image planes 101 and 102 were part of a prior downward sweep, thus the oldest image plane in this figure is plane 101. Each of the noncurrent image planes would thus have a greater transparency, or a lower opacity value associated with each of its pixels, than the next current one, the oldest image being most transparent. Thus, images significantly older than the current image will have reached an opacity of zero (or full transparency), and will have effectively completely faded away.

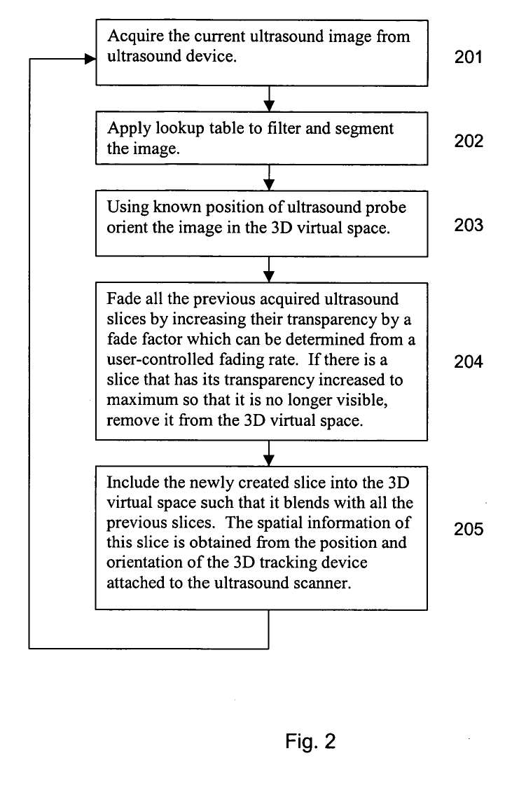

Process flow in an exemplary embodiment according to the present invention is depicted in FIG. 2. With reference thereto, at 201 a current ultrasound image is acquired from an ultrasound device. At 202 this image is processed according to a user defined color look-up table and the image is thus segmented. At 203, using the known position of the ultrasound probe the image is properly oriented in the virtual 3D space associated with the patient. At 204 all previously acquired ultrasound images are faded slices by increasing their transparency by a fade factor which can be determined by a user-controlled fading rate. If as a result there is a previous image that has its transparency increased to a maximum such that it is no longer visible, it is removed from the 3D virtual space at 204.

Finally, at 205 the newly created image is included into the 3D virtual space such that it blends with all the previous images. The spatial information associated with this newly created image (or “slice”) is obtained from the position and orientation of a 3D tracking device attached to the ultrasound scanner.

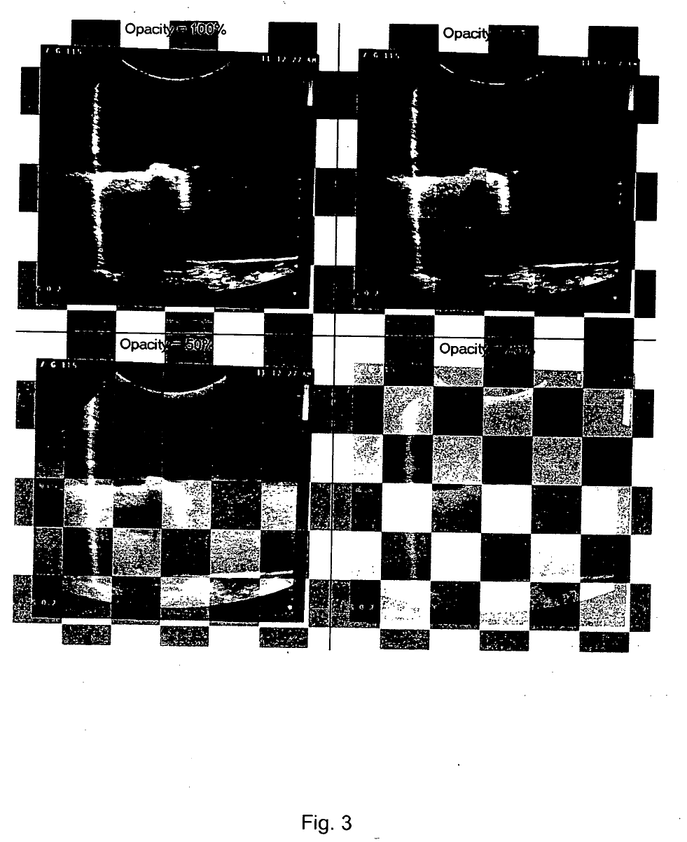

FIG. 3 depicts the same exemplary ultrasound image displayed with different opacities. In quadrant I the opacity is 100%, and none of the checkerboard background is visible. Quadrants II-IV show decreasing opacity of the image (and thus increasing transparency) such that the background is more and more visible in the combined image. Transparency is implemented by adding a pixel's intensity value multiplied by an opacity factor to an underlying pixel value. When three or more images of varying opacities are combined to form a resultant image, this addition is implemented recursively, according to techniques as are known in the art.

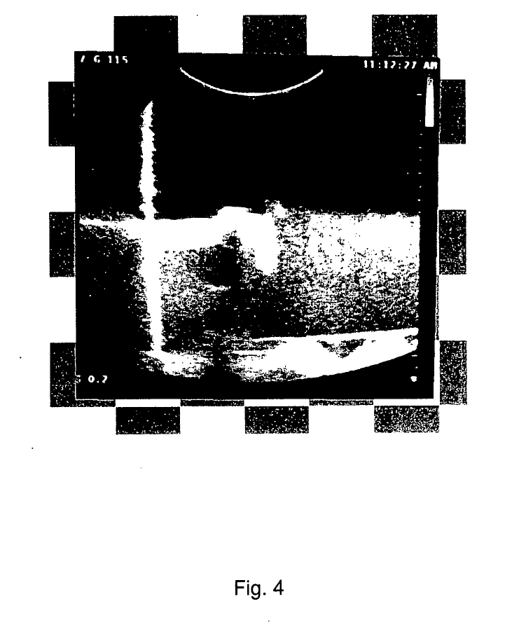

It is noted that changing the opacity of an image is different from changing its brightness. FIG. 4 shows the same image as shown in FIG. 3 with an opacity of 100% (as in FIG. 3, upper left quadrant) and with its brightness increased by 50% (relative to FIG. 3, upper left quadrant). It is noted that given the opacity of 100%, there is no blending with the checkerboard background, which thus cannot be seen through the image.

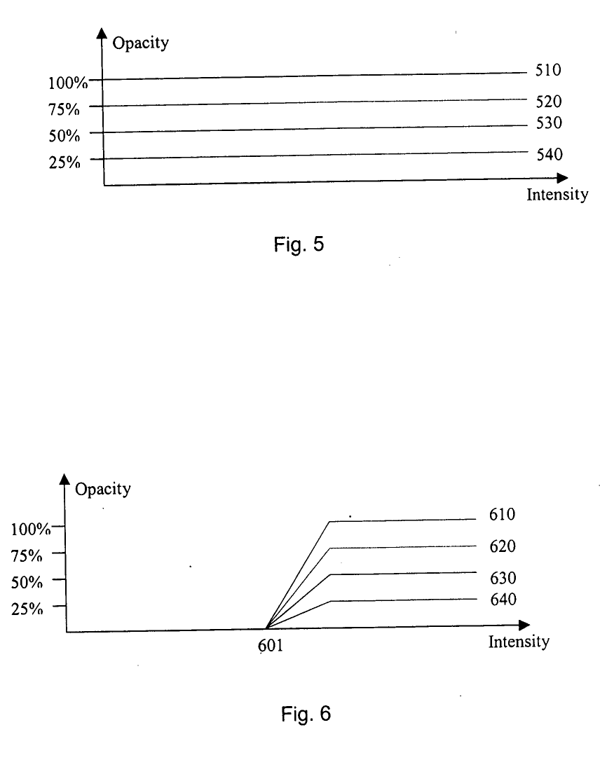

As depicted in each quadrant of FIG. 3, all of the pixels in an image have the same opacity value, regardless of their respective intensity. That is, whether a pixel is dark or bright, its opacity remains constant as shown on the opacity graphs depicted in FIG. 5. The different opacity vs. intensity plots in FIG. 5 correspond respectively to the images in each of the four quadrants of FIG. 3, as follows: 510=100% (upper left quadrant of FIG. 3), 520=75% (upper right quadrant), 530=50% (lower left quadrant) and 540=25% (lower right quadrant) opacity.

Alternatively, it is possible to vary the opacity of each of the pixels in an image as a function of their intensity. An example of such functions are the CLUTs described above. For example, darker pixels can be made less opaque and brighter pixels can, for example, be made more opaque, as is shown in the ramping up portions of the opacity vs. intensity plots depicted in FIG. 6. FIG. 7 depicts an example of using such a customized opacity table, or “customized CLUT.” It is noted that while one way to achieve this is a CLUT, it can also be done with an algorithm, using known techniques.

Fading

Fading is the process of decaying the opacity of an image over time. Thus, assuming for example that a given pixel has an opacity of α₀ at time t₀, and that in this example the maximum opacity (i.e., fully opaque) has a value of 1.0 and the minimum opacity (i.e., fully transparent) has a value of 0.0, then the opacity at an arbitrary time tn can be given by the equation:
αₙ=(1.0−f*n)α₀,
where f is the fading rate.

In general, a given “destination” pixel having a given greyscale intensity value I_{destination} in a given “destination” image can be blended with background or “source” pixel which underlies it having intensity value I_{source} and an opacity value α_{source} according to the following formulas:
Given: I_{source}[0-255], where [I=intensity]:
C_{source}=CLUT (I_{source}); (associates a color value with each greyscale intensity value according to a Color Look Up Table (“CLUT”)); and 1.
C_{combined}=C_{source}*α_{source}+(1−α_{source})*C_{destination}. 2.

Thus, in exemplary embodiments according to the present invention, using the fading rate as described above and recursively adding the acquired images in their temporal sequence using equations (1) and (2), a resultant display can be achieved.

Graphic Illustration

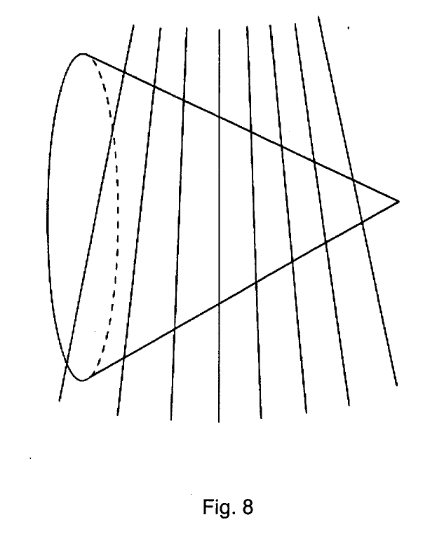

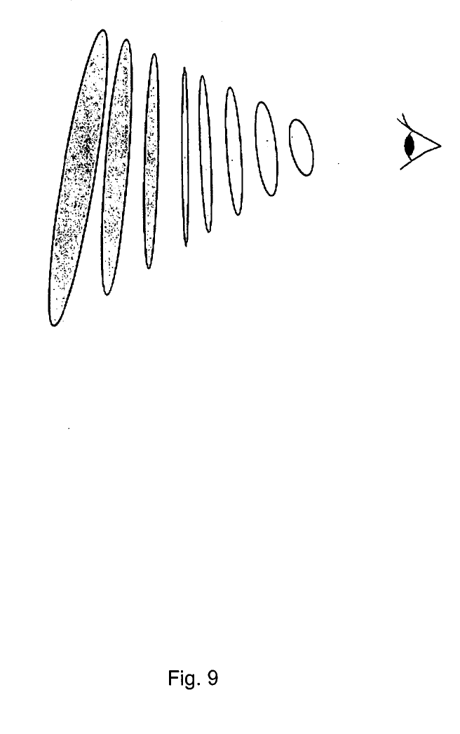

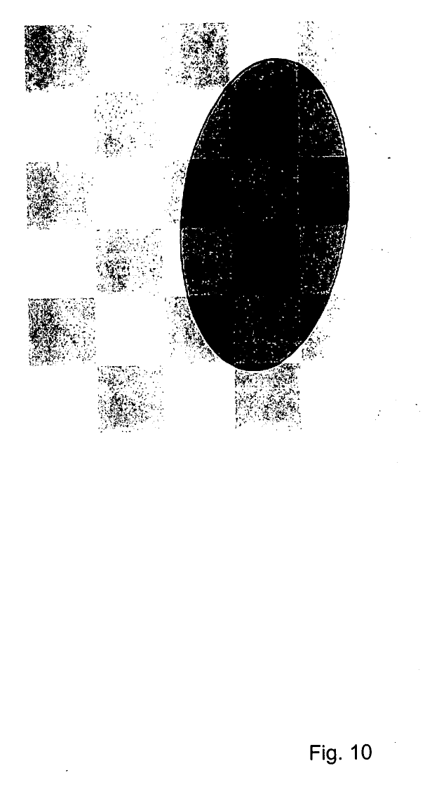

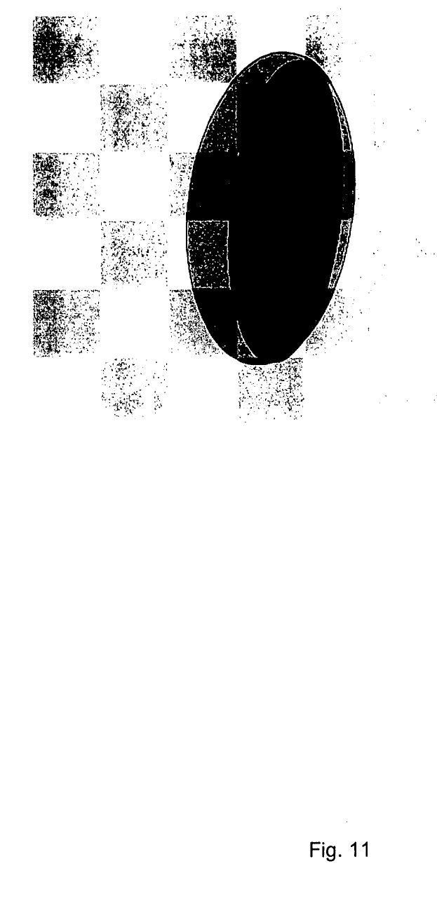

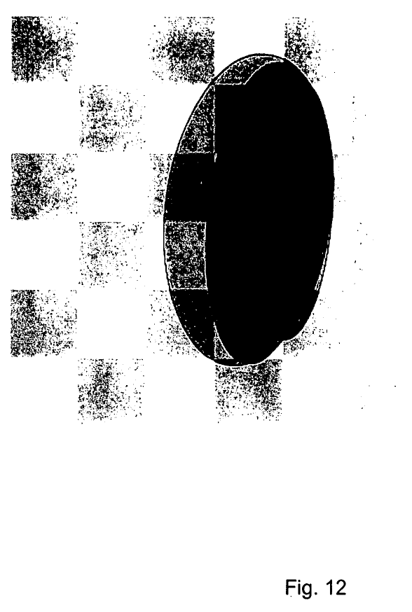

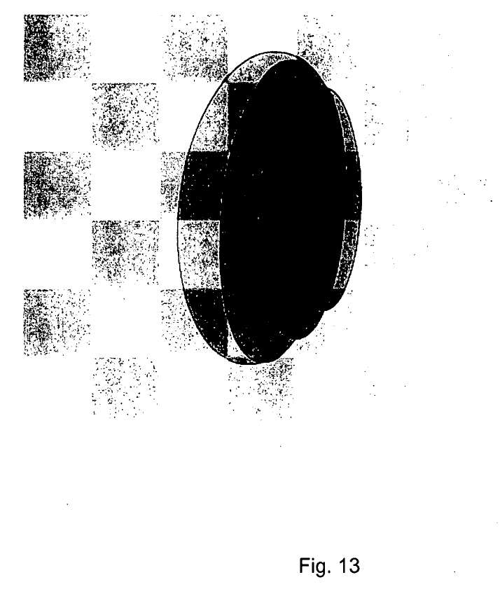

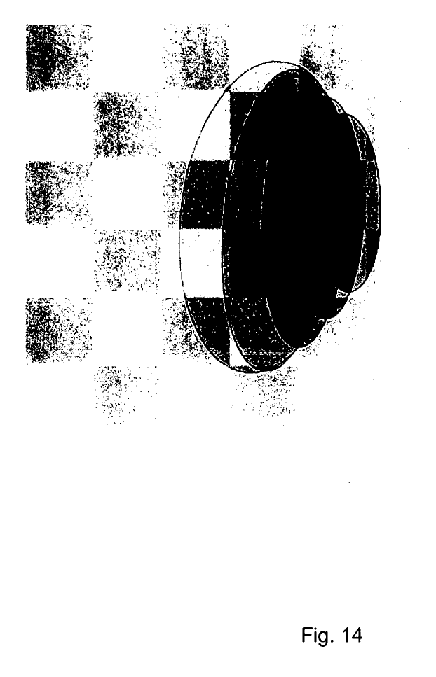

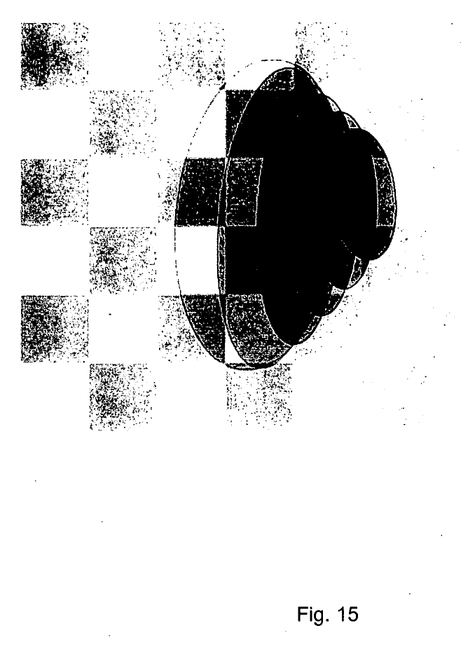

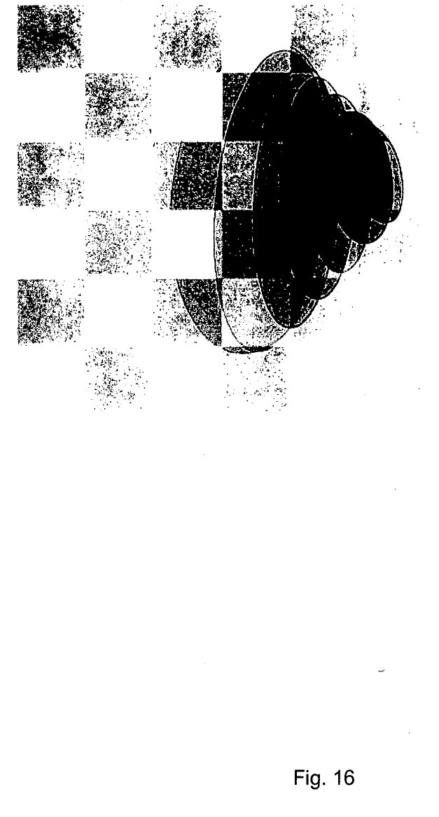

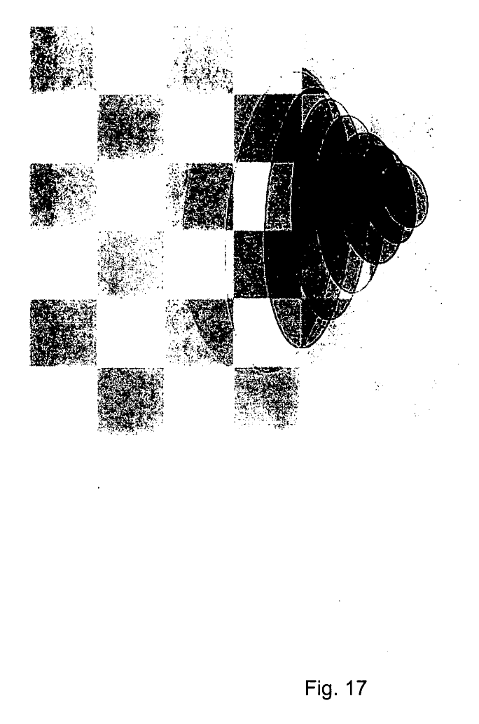

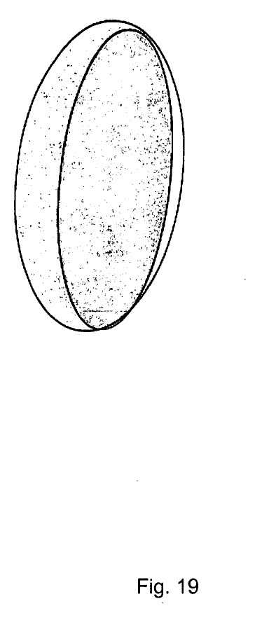

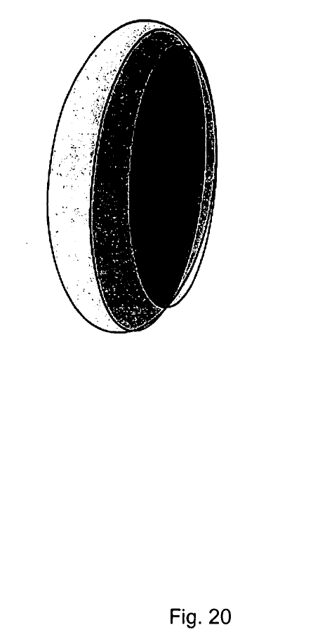

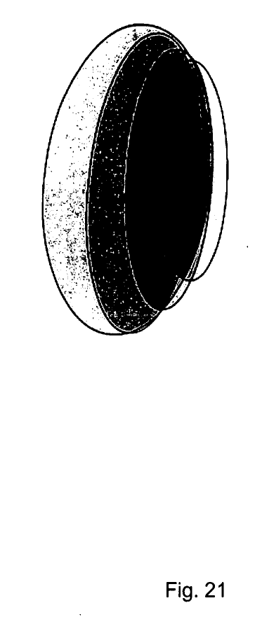

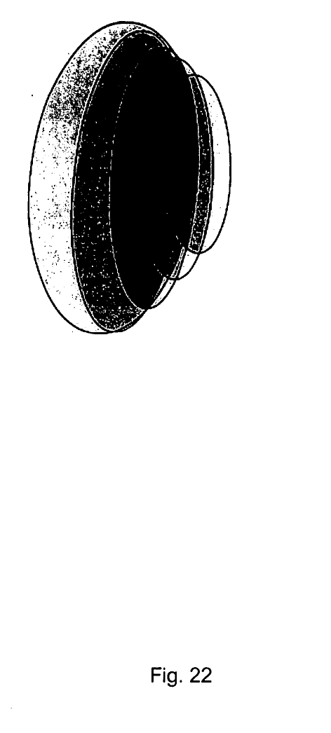

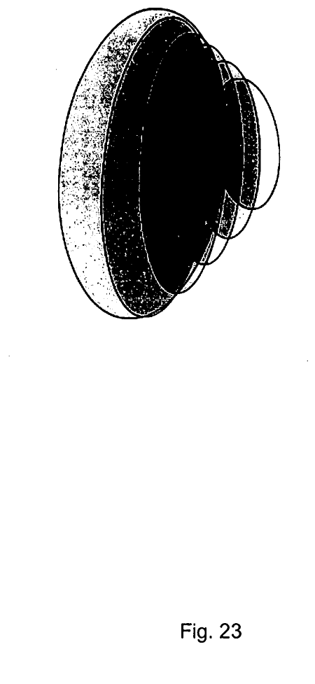

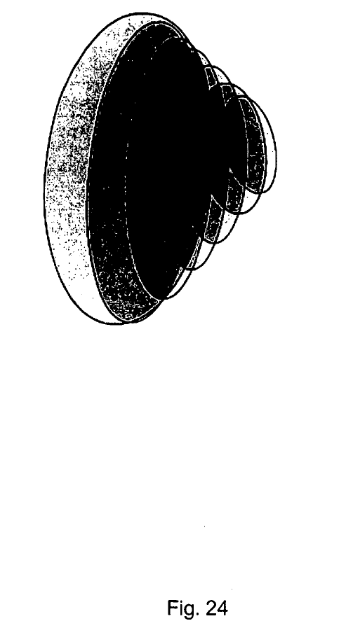

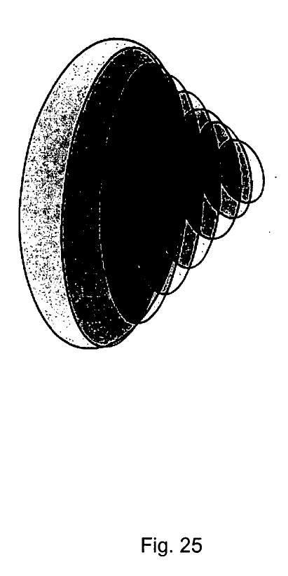

FIGS. 8 through 25 graphically illustrate methods according to exemplary embodiments of the present invention. A three dimensional cone is scanned with a plurality of probe positions along its longitudinal axis. It is noted that the viewpoint or perspective here is such that there is approximately a 45 degree angle between the viewpoint and a normal to the surface of the ovals. The acquired images are blended using a time dependent dissolving process, thus trace out a three-dimensional shape of the cone in real time. As each new (newer images are at the right of the figures, as the scan direction is from the left to the right in FIG. 8) image is acquired and displayed, older images have their respective transparencies increased until they simply fade away. The most current (rightmost) image in any figure is displayed with the greatest opacity, as described above.

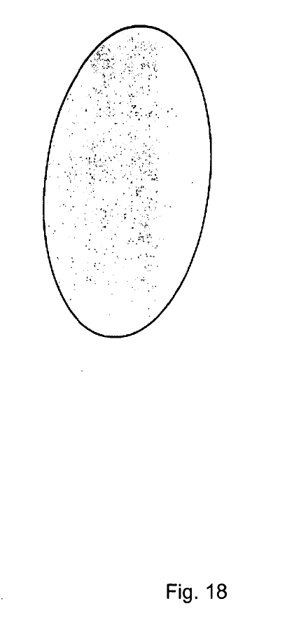

FIGS. 8-17 display the scan images over a checkerboard background, and FIGS. 18-25 display the same exemplary images over a plain white background.

Example Blended Images

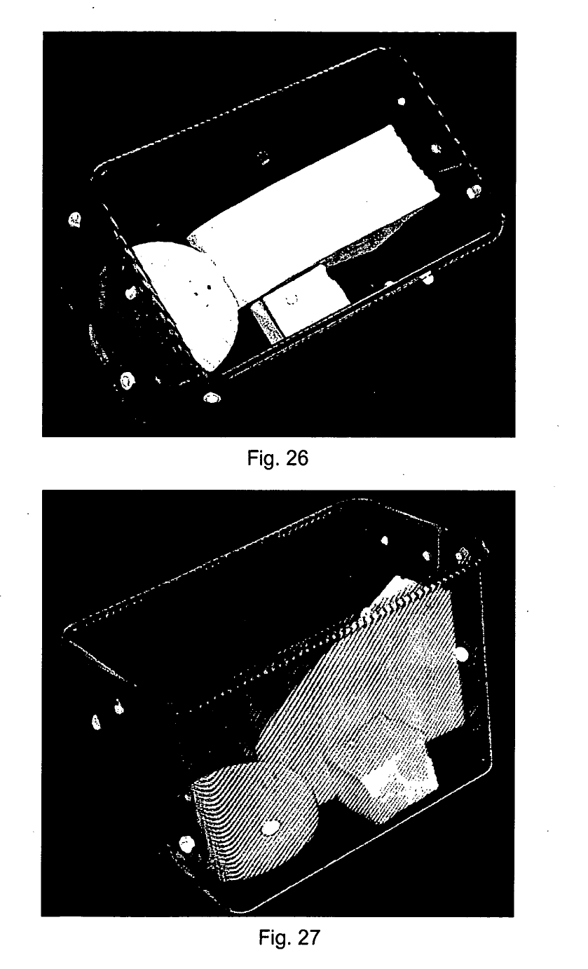

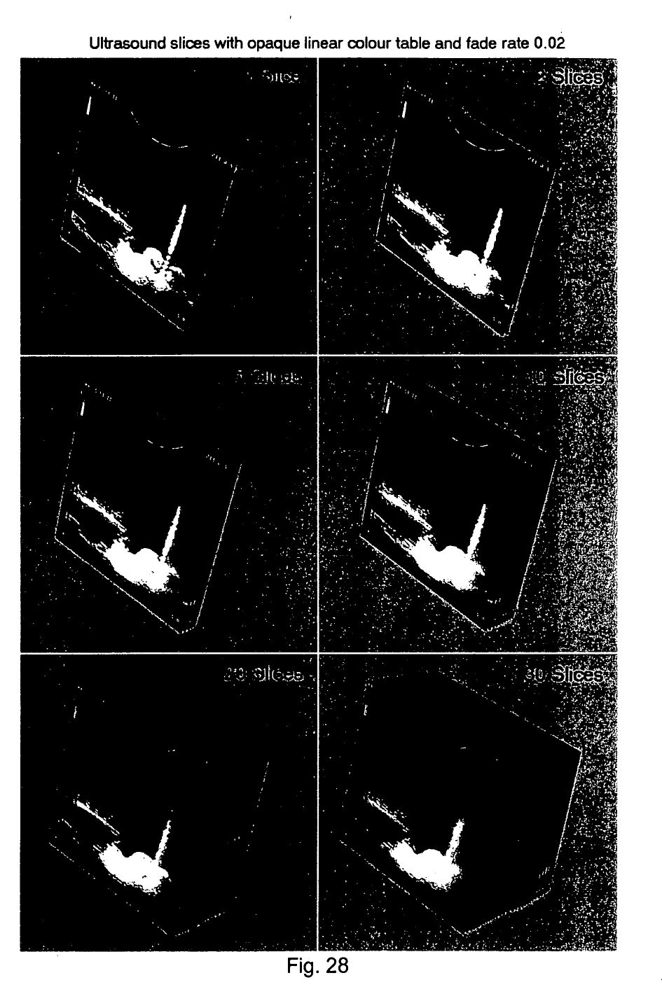

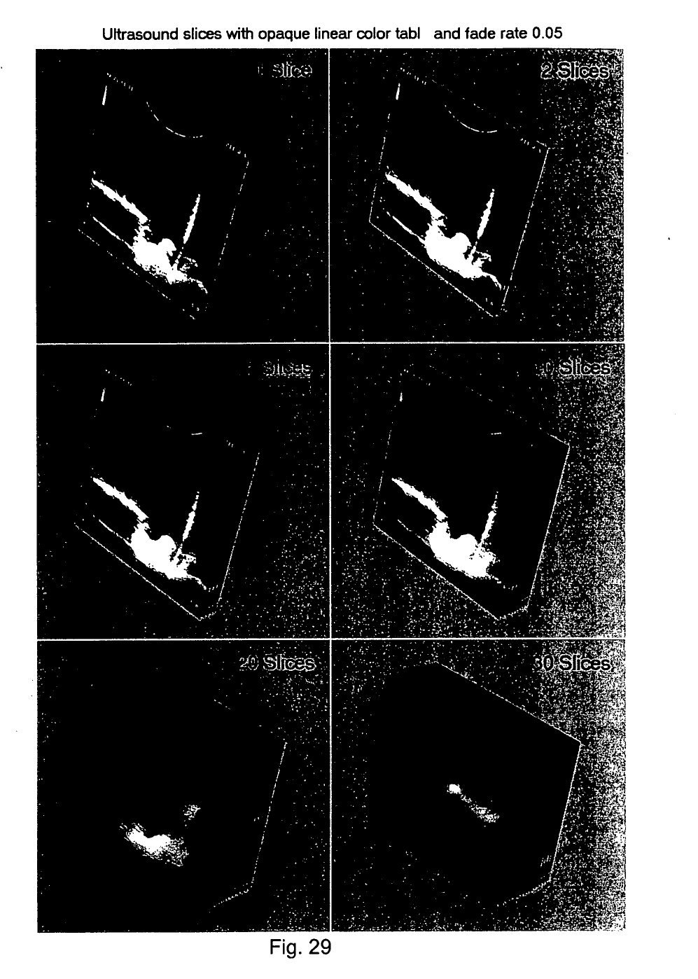

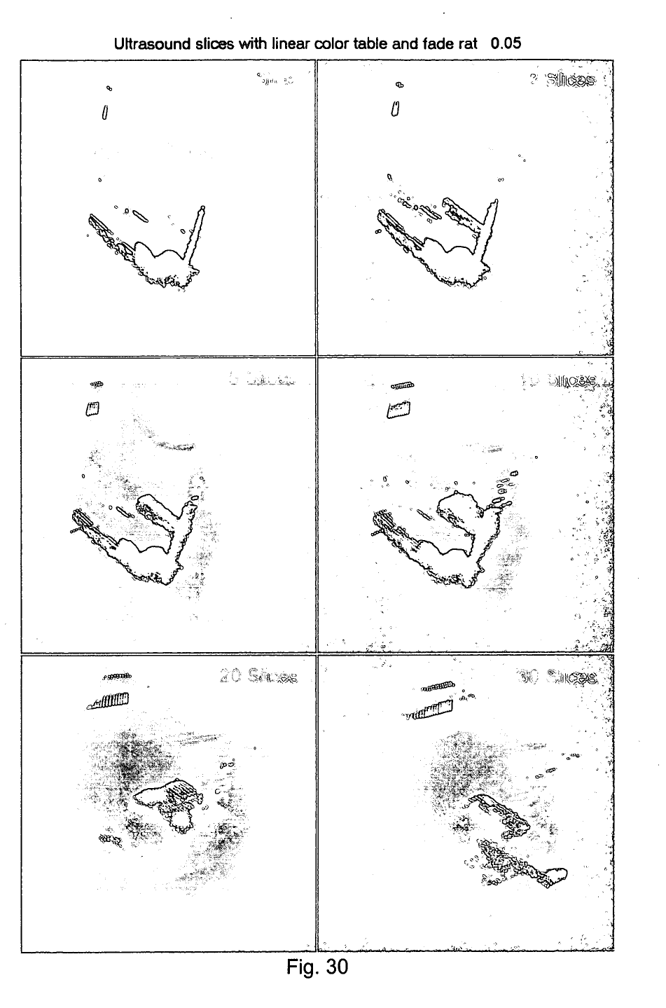

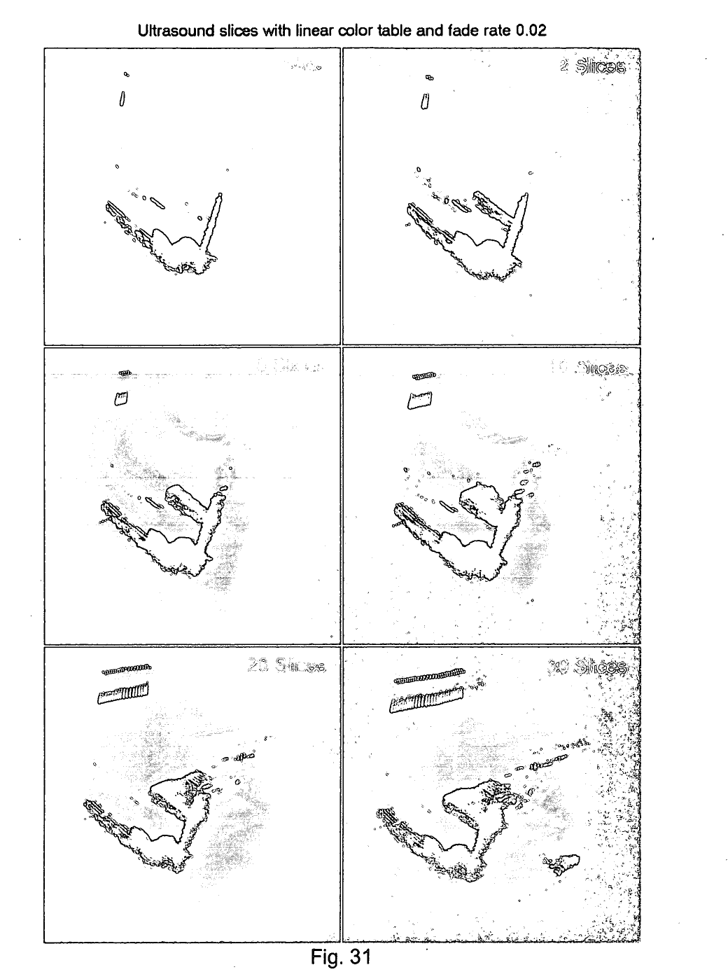

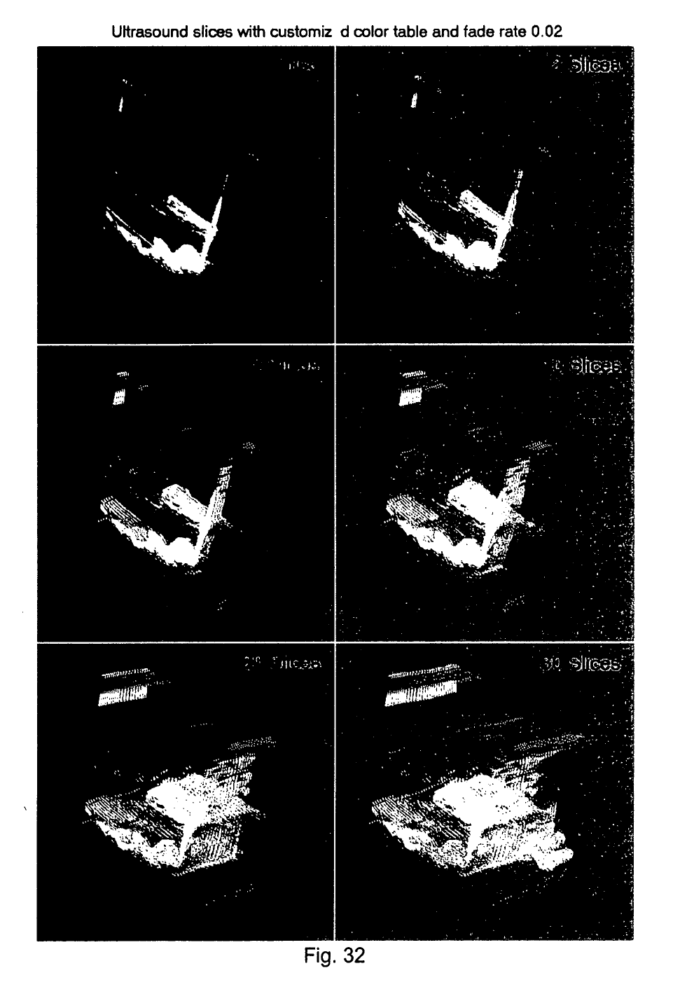

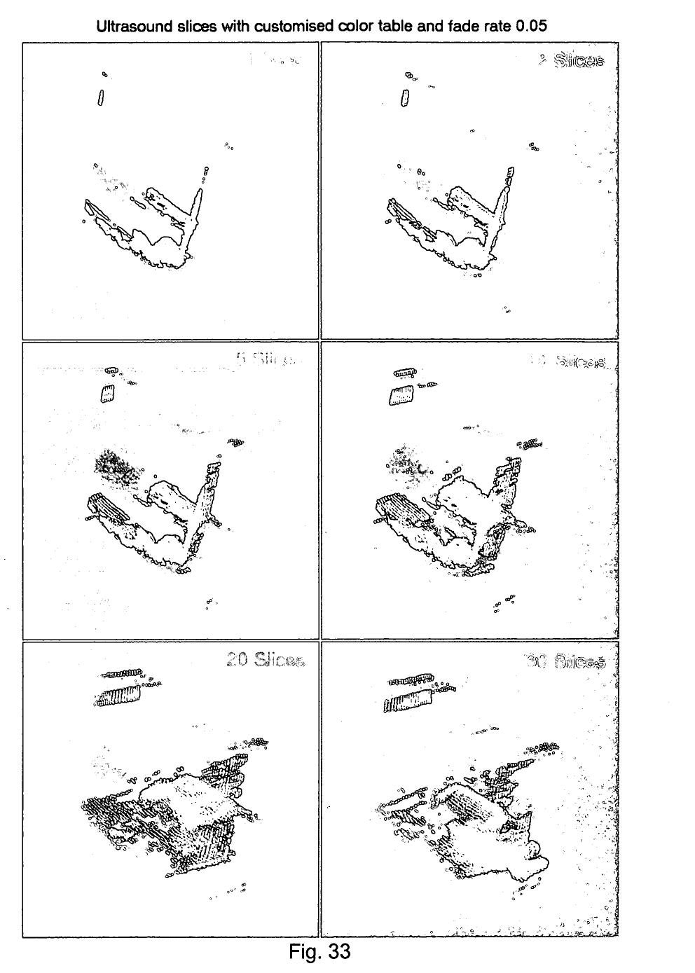

FIGS. 26 and 27 depict a CT scan of an exemplary set of phantom objects used to illustrate an exemplary embodiment according to the present invention. As can be seen in these figures, the phantom objects comprise a container containing three three-dimensional phantom objects. FIGS. 28 to 33 depict exemplary 3D ultrasound acquisitions of these objects. The exemplary acquisitions are done with different color look-up tables and different fading rates. FIG. 5 depicts the exemplary linear opaque color lookup table used fro FIGS. 28 and 29, FIG. 6A illustrates the exemplary “linear color look up table” used for FIGS. 30 and 31, and FIG. 6 depicts the exemplary “customized linear color look up table” used for FIGS. 32 and 33. For each combination of color look up table values and fade rates, exemplary blendings of 1, 2, 5, 10, 20 and 30 image slices are shown.

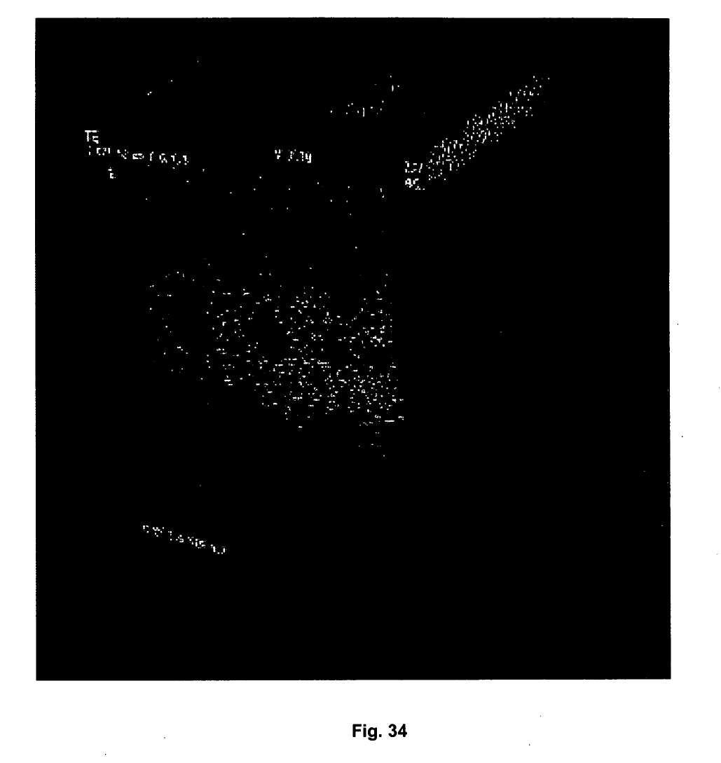

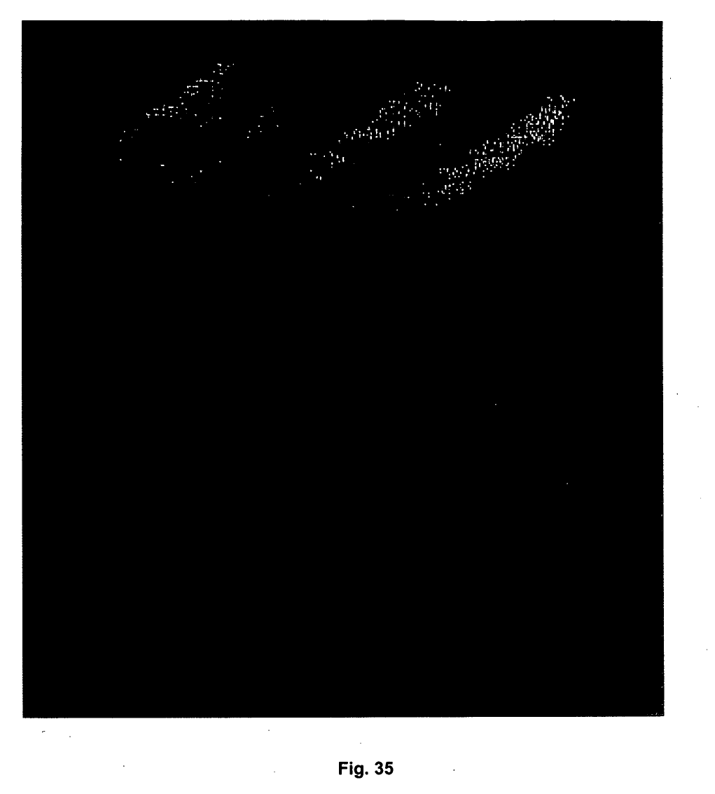

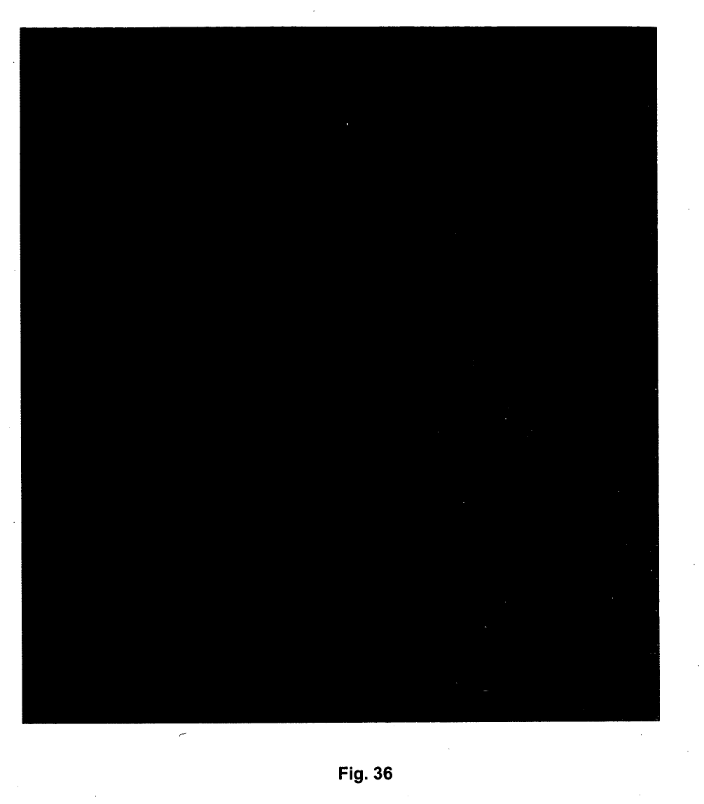

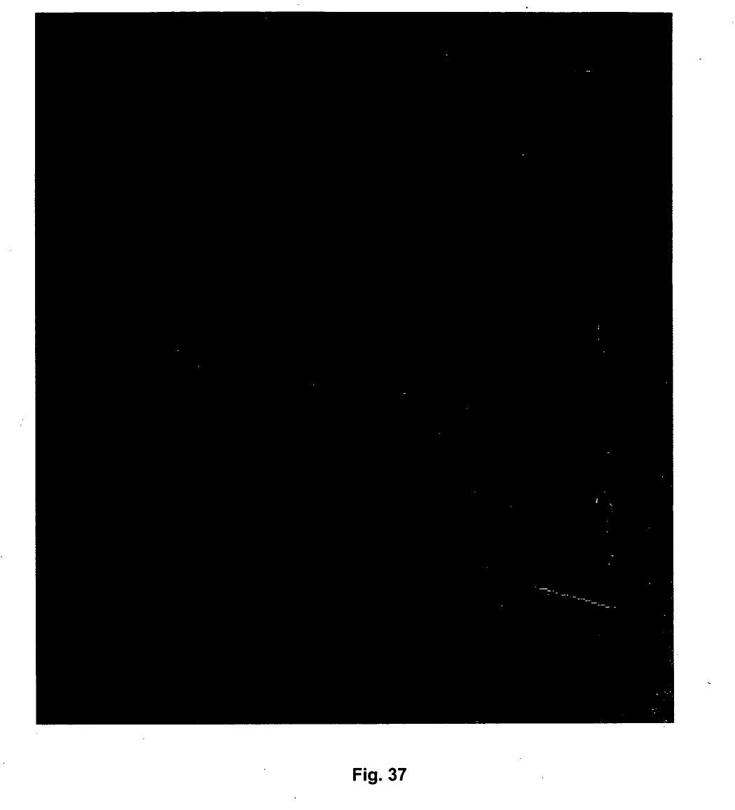

Additionally, FIGS. 34-37 depict blended ultrasound images of another type of phantom, according to an exemplary embodiment of the present invention. The phantom used to generate these images is essentially a box with a number of cylinders of different shapes, and placed at different locations, in it. In each of these images the ultrasound slices are blended from back to front, as described above. In each of these images the most current image is the one with the red boundary. Thus, in FIGS. 34 and 36 the user has swept towards the viewpoint (i.e. in the direction pointing up and out of the figures) such that the current slice is in front, and in FIGS. 35 and 37 the user has swept away from the viewpoint (i.e. in the direction pointing into the figures) such that the current slice is in back. Moreover, FIGS. 34-35 were filtered using a linear color look-up table, and FIGS. 36-37 were filtered using a customized color look-up table so that the darker cylinders are segmented out from their surroundings and given an orange hue. These variations illustrate some of the various perspectives a user can use to view an area of interest in an exemplary embodiment of the invention. Because all of these images are blended from back to front using the equations presented above, by viewing the objects using a backwards sweep (FIGS. 35 and 37) one can obtain a different point of view than by using a frontward sweep (FIGS. 34 and 36). As well, by filtering images using a customized CLUT a user can separate out structures of interest (FIGS. 36-37), and by using a linear CLUT (either invariant with intensity as depicted in FIG. 5, or variant with pixel intensity as depicted in FIG. 6) a user can view all of the area of interest as a whole.

In other exemplary embodiments of the present invention, various other blending schemes can be used, such as, for example, blending front to back. By using various fade rates, blending schema and CLUTs, in exemplary embodiments of the present invention the real time volumetric display effect can be adapted to various anatomical domains and various user preferences so as to convey the most information in the most efficient manner via an ultrasound examination.

Exemplary System Requirements

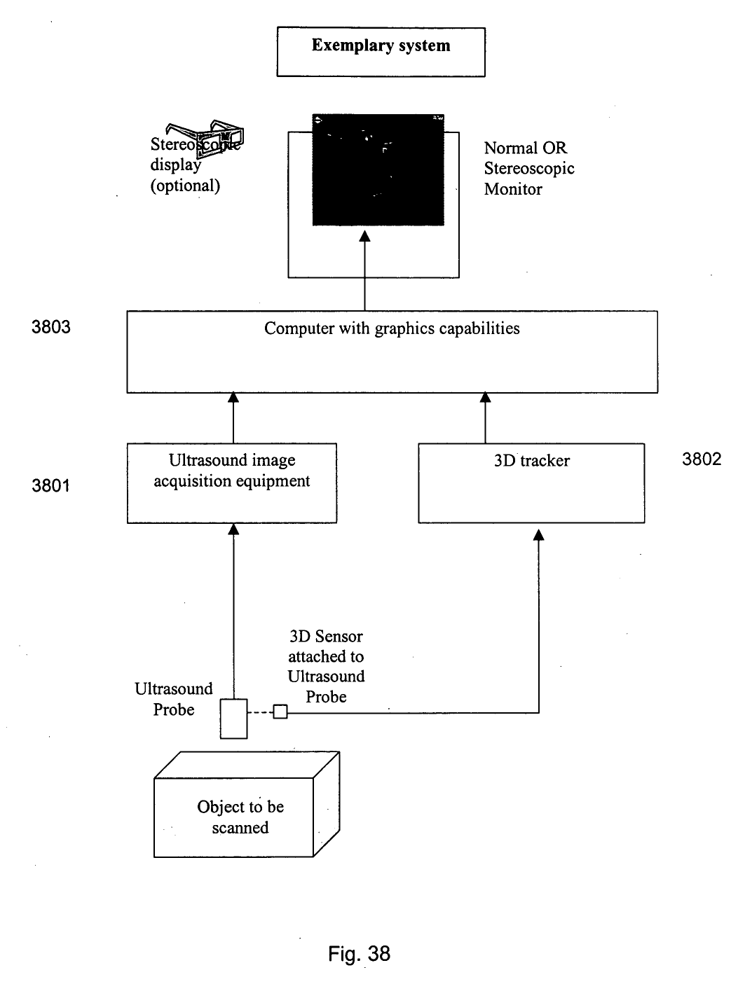

In exemplary embodiments according to the present invention, an exemplary system can comprise, for example, the following functional components with reference to FIG. 38:
1. An ultrasound image acquisition system 3801;
2. A 3D tracker 3802; and
3. A computer system with graphics capabilities 3803, to process an ultrasound image by combining it with the information provided by the tracker.

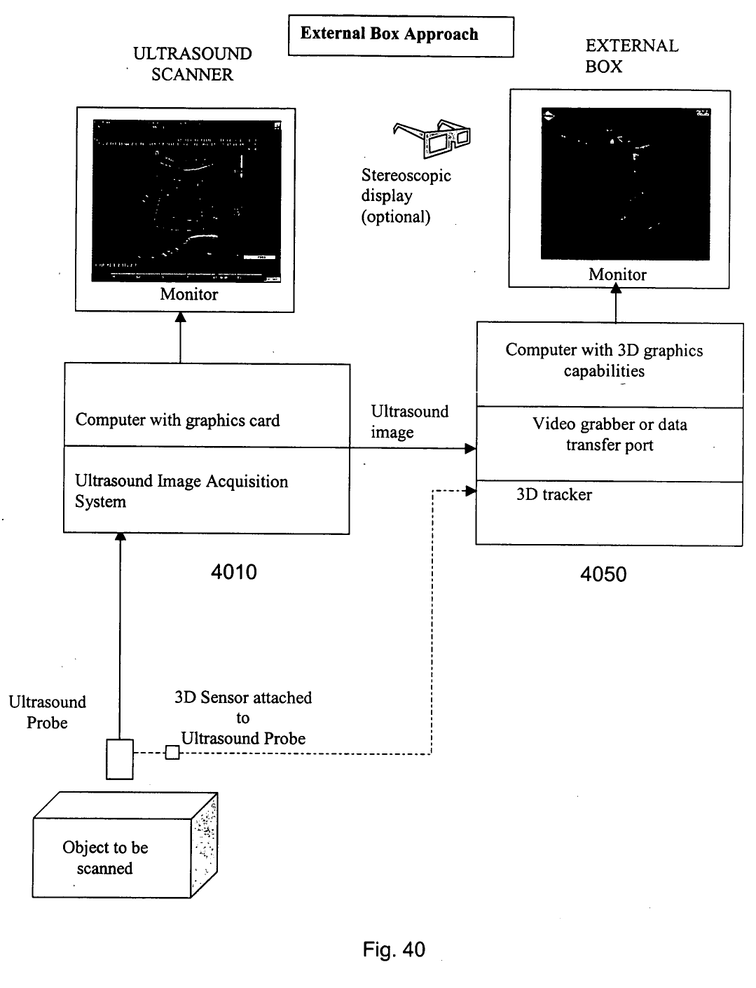

An exemplary system according to the present invention can take as input, for example, an analog video signal coming from an ultrasound scanner. This situation is illustrated, for example, in FIG. 40, where a standard ultrasound machine 4010 generates an ultrasound image and feeds it to a separate computer 4050 which then implements an exemplary embodiment of the present invention. A system can then, for example, produce as an output a 1024×768 VGA signal, or such other available resolution as may be desirable, which can be fed to a computer monitor for display. Alternatively, as noted below, an exemplary system can take as input a digital ultrasound signal.

Systems according to exemplary embodiments of the present invention can work either in monoscopic or stereoscopic modes, according to known techniques. In preferred exemplary embodiments according to the present invention, stereoscopy can be utilized inasmuch as it can significantly enhance the human understanding of images generated by this technique. This is due to the fact that stereoscopy can provide a fast and unequivocal way to discriminate depth.

Integration into Commercial Ultrasound Scanners

In exemplary embodiments according to the present invention, two options can be used to integrate systems implementing an exemplary embodiment of the present invention with existing ultrasound scanners:
1. Fully integrate functionality according to the present invention within an ultrasound scanner; or
2. Use an external box.

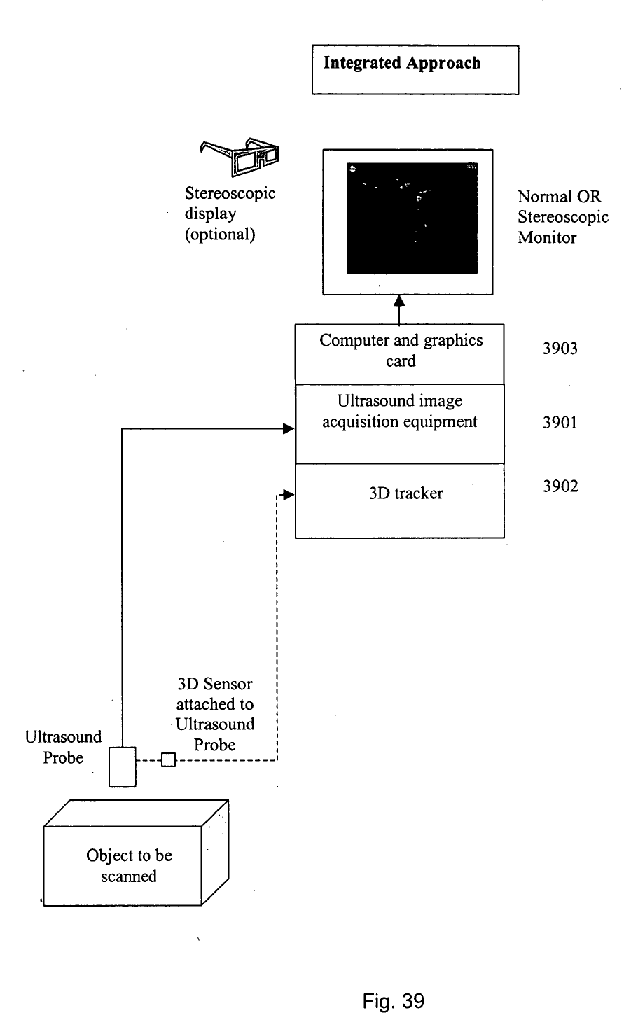

Each of these options will next be described, with reference to FIGS. 39 and 40, respectively.

Full Integration Option

In an exemplary fully integrated approach, with reference to FIG. 39, ultrasound image acquisition equipment 3901, a 3D tracker 3902 and a computer with graphics card 3903 are wholly integrated. In terms of real hardware, on a scanner such as, for example, the Technos MPX from Esaote S.p.A. (Genoa, Italy), full integration can easily be achieved, since such a scanner already provides most of the components required, except for a graphics card that supports the real-time blending of images. Additionally, as depicted in FIG. 39, optionally any stereoscopic display technique can be used, such as autostereoscopic displays, or anaglyphic red-green display techniques, using known techniques. A video grabber (not shown, but see FIG. 40) is also optional, and is in some exemplary embodiments undesired, since it would be best to provide as input to an exemplary system an original digital ultrasound signal. However, in other exemplary embodiments of the present invention it may be economical to use an analog signal since that is what is generally available in existing ultrasound systems. A fuilly integrated approach, such as is depicted in FIG. 39, can, for example, take full advantage of a digital ultrasound signal.

External Box Option

This approach requires a box external to the ultrasound scanner that takes as an input the ultrasound image (either as a standard video signal or as a digital image), and provides as an output a 3D display. This is reflected in the exemplary system depicted in FIG. 40. Such an external box can, for example, connect through a video analog signal. As noted, this is not an ideal solution, since scanner information such as, for example, depth, focus, etc., would have to be obtained by image processing on the text displayed in the video signal. Such processing would have to be customized for each scanner model, and would be subject to modifications in the user interface of the scanner. A better approach, for example, is to obtain this information via a data digital link, such as, for example, a USB port, or a network port. An external box can be, for example, a computer with two PCI slots, one for the video grabber (or a data transfer port capable of accepting the ultrasound digital image) and another for the 3D tracker.

The present invention has been described in connection with exemplary embodiments and implementations, as examples only. It is understood by those having ordinary skill in the pertinent arts that modifications to any of the exemplary embodiments or implementations can be easily made without materially departing from the scope or spirit of the present invention, which is defined by the appended claims.

1. A method for dynamic three dimensional display of ultrasound images, comprising:
acquiring a plurality of ultrasound images with a probe;
tracking the three-dimensional position of the probe as each image is acquired;
blending the plurality of ultrasound images in substantially real time using the three-dimensional positional information; and
displaying the combined image on a display.

2. The method of claim 1, wherein said blending includes adding successive images according to a time dependent dissolving process;

3. The method of claim 2, wherein said time dependent dissolving process includes decaying the opacity of an image over time.

4. The method of claim 1 wherein the acquired ultrasound images are blended from front to back.

5. The method of claim 1 wherein the acquired ultrasound images are blended from back to front.

6. The method of claim 3, wherein said decaying the opacity includes calculating the opacity αₙ at time tₙ by the following equation: αₙ=(1.0−f*n)α₀, where f is a fading rate.

7. The method of claim 6, wherein f is in the range of 0.01 to 0.10.

8. The method of claim 6, wherein f is in the range of 0.01 to 0.10.

9. The method of claim 3, wherein the opacity value of a pixel in a given image can vary with its intensity according to predetermined parameters.

10. The method of claim 3, wherein the opacity value of a pixel in a given image can vary with its intensity according to user defined parameters.

11. The method of claim 3, where all pixels in a given image have the same opacity value.

12. The method of claim 1, where prior to being blended, each image is filtered using a color lookup table.

13. The method of claim 1, where the combined image is displayed stereoscopically.

14. A system for displaying ultrasound images pseudo-volumetrically, comprising:
an ultrasound image acquisition system including a probe;
a tracking system arranged to track the probe;
a computer system arranged to process acquired ultrasound images utilizing information provided by the tracking system; and
a 3D display arranged to display the processed ultrasound images.

15. The system of claim 14, wherein said computer system blends the ultrasound images by adding successive images according to a time dependent dissolving process;

16. The system of claim 15, wherein said time dependent dissolving process includes decaying the opacity of an image over time.

17. The system of claim 15 wherein the acquired ultrasound images are blended from back to front.

18. The system of claim 14, wherein the ultrasound image acquisition system, the tracking system and the computer system are all integrated within a single system.

19. The system of claim 14, wherein the ultrasound image acquisition system provides an image signal to an external system, wherein the external system comprises the tracking system, the computer system and the display.

20. The system of claim 14, wherein the display is stereoscopic.

21. The system of claim 19, wherein the system is stereoscopic.

22. The system of claim 14, wherein the acquired ultrasound images are blended from front to back.

23. The system of claim 16, wherein said decaying the opacity includes calculating the opacity αₙ at time tₙ by the following equation: αₙ=(1.0−f*n)α₀, where f is a fading rate.

24. The method of claim 12, wherein the color look-up table is one of linear, opaque, customized linear or customized opaque.

25. The method of claim 1, wherein an indication of the position, orientation or extent of a noncurrent image is displayed without displaying the noncurrent image.
